(19) United States
(12) Patent Application Publication
(10) Pub. No.: US 2018/0070921 A1
(43) Pub. Date: Mar. 15, 2018

(54) A METHOD AND APPARATUS FOR PERFORMING MEASUREMENT ON AN M-MODE ULTRASONIC IMAGE

(71) Applicant: GE MEDICAL SYSTEMS GLOBAL TECHNOLOGY COMPANY, LLC, WAUKESHA, WI (US)

(72) Inventors: Gang Liu, Wuxi (CN); Zhaodong Jiang, Wuxi (CN); Hujie Jiang, Wuxi (CN); Dechun Huang, NanNing (CN); Yulei Gao, Wuxi (CN)

(73) Assignee: GENERAL ELECTRIC COMPANY, Schenectady, NY (US)

(21) Appl. No.: 15/560,037

(22) PCT Filed: Mar. 20, 2015
(86) PCT No.: PCT/CN2015/074692

§ 371 Date: Sep. 20, 2017

(51) Int. Cl. A61B 8/08; A61B 8/14; G06T 7/536; G01N 29/22

(52) U.S. Cl. CPC A61B 8/486; G01N 29/22; G06T 7/536; A61B 8/14

(57) ABSTRACT

The present invention relates to a method and apparatus for performing measurement on an M-mode ultrasonic image. The method comprises: obtaining position information of measurement points on the M-mode ultrasonic image; marking corresponding points of the measurement points on a B-mode ultrasonic image corresponding to the M-mode ultrasonic image based on the position information; and performing measurement on the M-mode ultrasonic image based on the position information.

### FIELD

The present invention relates to a method and apparatus for performing measurement on an ultrasonic image, particularly to a method and apparatus for performing measurement on an M-mode ultrasonic image.

### BACKGROUND

Ultrasonic images usually have two types, i.e., B-mode and M-mode. For a B-mode ultrasonic image, ultrasonic echo signals are displayed in form of light spots. If the echo is strong, the light spot is bright; if the echo is weak, the light spot is dark. The B-mode ultrasonic image may show an anatomic section of viscera. For an M-mode ultrasonic image, a slow scan saw-tooth wave is added on the basis of a single-beam B-type scan, converting light spots into a curve so that the echo light spots automatically move and scan from left to right.

An M-mode ultrasonic image may be used for measuring some length information that will change with time, particularly used for measuring some parameters of organs such as hear, blood vessel and the like, for example, left ventricular ejection fraction (LVEF), interventricular septal thickness (IVS), etc.

The existing method for performing measurement on an M-mode ultrasonic image is done by observing the M-mode ultrasonic image with naked eyes and then manually choosing suitable measurement points on the M-mode ultrasonic image by an ultrasonography department doctor. Since the M-mode ultrasonic image does not include anatomic information of viscera, it is very difficult for an inexperienced ultrasonography department doctor to choose measurement points accurately.

### SUMMARY

An objective of the present invention is to provide a method and apparatus for performing measurement on an M-mode ultrasonic image, capable of guiding the doctor to complete the measurement on the M-mode ultrasonic image rapidly and accurately.

One embodiment of the present invention provides a method for performing measurement on an M-mode ultrasonic image, comprising: obtaining position information of measurement points on the M-mode ultrasonic image; marking corresponding points of the measurement points on a B-mode ultrasonic image corresponding to the M-mode ultrasonic image based on the position information; and performing measurement on the M-mode ultrasonic image based on the position information.

Another embodiment of the present invention provides an apparatus for performing measurement on an M-mode ultrasonic image, comprising: an obtaining module for obtaining position information of measurement points on the M-mode ultrasonic image; a marking module for marking corresponding points of the measurement points on a B-mode ultrasonic image corresponding to the M-mode ultrasonic image based on the position information; and a measuring module for performing measurement on the M-mode ultrasonic image based on the position information.

### BRIEF DESCRIPTION OF THE DRAWINGS

The present invention can be better understood in light of the following description of embodiments of the present invention with reference to the accompanying drawings, in which:

FIG. 1 is a general flow schematic diagram of one embodiment of a method for performing measurement on an M-mode ultrasonic image of the present invention;

FIG. 2 shows a flow schematic diagram of one embodiment for marking corresponding points of the measurement points on a B-mode ultrasonic image corresponding to the M-mode ultrasonic image based on the position information during performing measurement on the M-mode ultrasonic image of the present invention;

FIG. 3 shows a schematic block diagram of one embodiment of an apparatus for performing measurement on an M-mode ultrasonic image of the present invention.

### DETAILED DESCRIPTION

Hereafter, a detailed description will be given for preferred embodiments of the present invention. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art relating to the contents disclosed in the present invention, which should not be regarded as insufficient disclosure of the present invention.

Unless defined otherwise, all the technical or scientific terms used in the Claims and the Description should have the same meanings as commonly understood by one of ordinary skilled in the art to which the present invention belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

In order to make the purpose, the technical solutions and the advantages of the invention more apparent, the technical solutions of the present invention will be set forth clearly and fully in the following by combining with specific embodiments of the invention and the corresponding accompanying drawings. Obviously, the described embodiments are merely part—not all—of the embodiments in the present invention. In view of the embodiments in the present invention, other embodiments made by one of ordinary skilled in the art without inventive work all fall within the scope of protection of the invention.

According to the embodiments of the present invention, a method for performing measurement on an M-mode ultrasonic image is provided.

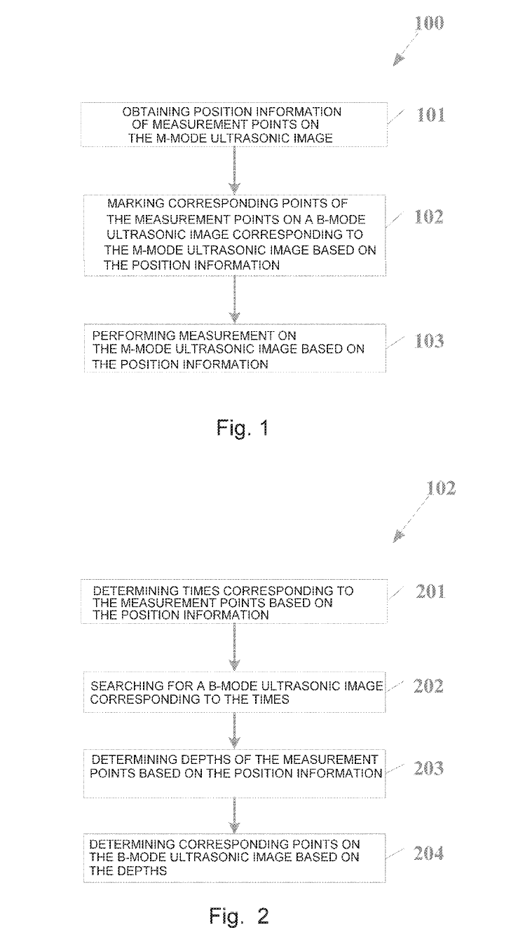

Refer to FIG. 1, which shows a flow schematic diagram of one embodiment of a method 100 for performing measurement on an M-mode ultrasonic image of the present invention. The method 100 may comprise the following Steps 101 to 103.

As shown in FIG. 1, in Step 101, position information of measurement points on the M-mode ultrasonic image is obtained.

When performing an ultrasonic scan, the doctor may mark points for measurement on the generated M-mode ultrasonic image, which are referred to as measurement points.

In one embodiment of the present invention, when the doctor marks one measurement point on the M-mode ultrasonic image, position information of the measurement point may be obtained by the abscissa and the ordinate values of the measurement point.

In Step 102, the corresponding points of the measurement points are marked on a B-mode ultrasonic image corresponding to the M-mode ultrasonic image based on the position information.

In one embodiment of the present invention, positions of the measurement points on the B-mode ultrasonic image corresponding to the M-mode ultrasonic image may be obtained based on the position information of the measurement points on the M-mode ultrasonic image obtained in Step 101, thereby marking the measurement points on the B-mode ultrasonic image.

Refer to FIG. 2, which shows a flow schematic diagram of one embodiment of Step 102. Step 102 may comprise the following sub-Steps 201 to 204.

In sub-Step 201, times corresponding to the measurement points are determined based on the position information.

In one embodiment of the present invention, the time corresponding to the measurement point may be represented by the abscissa in the position information of the measurement point on the M-mode ultrasonic image.

In sub-Step 202, a B-mode ultrasonic image corresponding to the time is searched.

In one embodiment of the present invention, based on the time determined by sub-Step 101, a B-mode ultrasonic image corresponding to that time may be searched out among the stored B-mode ultrasonic images.

In sub-Step 203, a depth of the measurement point is determined based on the position information.

In one embodiment of the present invention, the depth of the measurement point may be determined by the ordinate in the position information of the measurement point on the M-mode ultrasonic image.

In sub-Step 204, a corresponding point is determined on the B-mode ultrasonic image based on the depth.

In one embodiment of the present invention, the position of the corresponding point of the measurement point on the B-mode ultrasonic image may be determined by an appropriate coordinate transform on the depth of the measurement point obtained in sub-Step 203, thus marking the corresponding point on the B-mode ultrasonic image determined by sub-Step 202.

After Step 102, the doctor may see the position of each measurement point, which is chosen by himself on the M-mode ultrasonic image, on the corresponding B-mode ultrasonic image, and may in turn judge whether the positions of these measurement points are suitable or not by combining with the anatomic information on the B-mode ultrasonic image. If the doctor believes that the position of a certain measurement point is not suitable, a measurement point may be re-chosen on the M-mode ultrasonic image, i.e., cancelling the existing measurement point and selecting one new measurement point. This may be regarded as changing the position of one measurement point. In other words, when a certain measurement point is cancelled from the M-mode ultrasonic image, correspondingly, its corresponding point on the B-mode ultrasonic image is also cancelled, then a corresponding point of a new measurement point may be re-marked on the B-mode ultrasonic image by re-performing Step 102, until the doctor considers that the positions of the measurement points have satisfied the requirements.

In Step 103, measurement is performed on the M-mode ultrasonic image based on the position information.

In one embodiment of the present invention, if an object to be measured is a length of one segment of straight line on the M-mode ultrasonic image, when the number of the measurement points reaches two, a straight distance between the two measurement points may be calculated on the M-mode ultrasonic image, thereby completing one measurement. Moreover, the measurement result may also be displayed on the corresponding B-mode ultrasonic image.

In one embodiment of the present invention, after one measurement is completed, the measurement points involved in the measurement may not be marked on the M-mode ultrasonic image any more, and also the corresponding points of these measurement points are not marked on the B-mode ultrasonic image any more.

So far a method for performing measurement on an M-mode ultrasonic image according to the embodiments of the present invention has been described. When the doctor sets/updates measurement points on the M-mode ultrasonic image, the method according to the present invention can automatically display/update the positions of the measurement points on the corresponding B-mode ultrasonic image, so that the doctor may conveniently judge whether the measurement points are set in suitable positions. In this way, speed and accuracy for performing measurement on the M-mode ultrasonic image may be improved.

Similar to the method, the present invention also provides a corresponding apparatus.

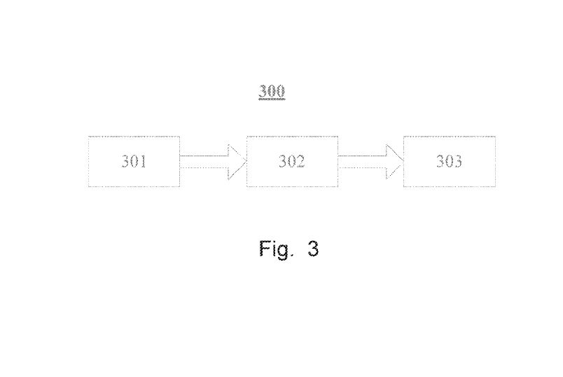

FIG. 3 shows a schematic block diagram of one embodiment of an apparatus 300 for performing measurement on an M-mode ultrasonic image of the present invention.

As shown in FIG. 3, the apparatus 300 may comprise: an obtaining module 301 for obtaining position information of measurement points on the M-mode ultrasonic image; a marking module 302 for marking corresponding points of the measurement points on a B-mode ultrasonic image corresponding to the M-mode ultrasonic image based on the position information; and a measuring module 303 for performing measurement on the M-mode ultrasonic image based on the position information.

In one embodiment of the present invention, the marking module 302 may further comprise a time-determining module for determining times corresponding to the measurement points based on the position information; and a searching module for searching for the B-mode ultrasonic image corresponding to the times.

In one embodiment of the present invention, the marking module 302 may further comprise: a depth-determining module for determining depths of the measurement points based on the position information; and a corresponding-point-determining module for determining the corresponding points on the B-mode ultrasonic image based on the depths.

In one embodiment of the present invention, the apparatus 300 may further comprise: a re-marking module for re-marking the corresponding points on the B-mode ultrasonic image when the position changes.

In one embodiment of the present invention, the measuring module 303 may further comprise: a distance-calculating module for calculating a straight distance between two measurement points on the M-mode ultrasonic image when the number of the measurement points reaches two.

So far an apparatus for performing measurement on an M-mode ultrasonic image according to the embodiments of the present invention has been described. Similar to the above method, when the doctor sets/updates measurement points on the M-mode ultrasonic image, the apparatus according to the present invention can automatically display/update the positions of the measurement points on the corresponding B-mode ultrasonic image, so that the doctor may conveniently judge whether the measurement points are set in suitable positions. In this way, speed and accuracy for performing measurement on the M-mode ultrasonic image may be improved. Moreover, the technical solution of the present invention may be applied to various M-modes, for example, Basic M-mode, Anatomic M-mode, Curved Anatomical M-mode and the like.

The above descriptions are merely embodiments of the invention and are not intended to restrict the scope of the invention. All kinds of variations and modifications could be made to the present invention to those skilled in the art. Any modifications, alternatives and improvements made within the spirit and principles of the present invention shall fall within the scope of the appended claims.

What is claimed is:

1. A method for performing measurement on an M-mode ultrasonic image, comprising:
obtaining position information of measurement points on said M-mode ultrasonic image;
marking corresponding points of the measurement points on a B-mode ultrasonic image corresponding to said M-mode ultrasonic image based on said position information; and
performing measurement on the M-mode ultrasonic image based on said position information.

2. The method according to claim 1, wherein the step of marking corresponding points of the measurement points on a B-mode ultrasonic image corresponding to said M-mode ultrasonic image based on said position information further comprises:
determining times corresponding to the measurement points based on said position information; and
searching for the B-mode ultrasonic image corresponding to the times.

3. The method according to claim 2, wherein the step of marking corresponding points of the measurement points on a B-mode ultrasonic image corresponding to said M-mode ultrasonic image based on said position information further comprises:
determining depths of the measurement points based on said position information; and
determining the corresponding points on said B-mode ultrasonic image based on the depths.

4. The method according to claim 1, further comprising:
re-marking the corresponding points on said B-mode ultrasonic image when said position information changes.

5. The method according to claim 1, wherein the step of performing measurement on the M-mode ultrasonic image based on said position information further comprises:
calculating a straight distance between two of said measurement points on the M-mode ultrasonic image when the number of said measurement points reaches two.

6. An apparatus for performing measurement on an M-mode ultrasonic image, comprising:
an obtaining module for obtaining position information of measurement points on said M-mode ultrasonic image;
a marking module for marking corresponding points of the measurement points on a B-mode ultrasonic image corresponding to said M-mode ultrasonic image based on said position information; and
a measuring module for performing measurement on said M-mode ultrasonic image based on said position information.

7. The apparatus according to claim 6, wherein the marking module further comprises:
a time-determining module for determining times corresponding to the measurement points based on said position information; and
a searching module for searching for the B-mode ultrasonic image corresponding to the times.

8. The apparatus according to claim 7, wherein the marking module further comprises:
a depth-determining module for determining depths of the measurement points based on said position information; and
a corresponding-point-determining module for determining the corresponding points on said B-mode ultrasonic image based on the depths.

9. The apparatus according to claim 6, further comprising:
a re-marking module for re-marking the corresponding points on said B-mode ultrasonic image when said position information changes.

10. The apparatus according to claim 6, wherein the measuring module further comprises:
a distance-calculating module for calculating a straight distance between two of said measurement points on the M-mode ultrasonic image when the number of said measurement points reaches two.
